(19) United States
(12) Patent Application Publication
(10) Pub. No.: US 2007/0207097 A1
(43) Pub. Date: Sep. 6, 2007

(54) Treating hair or nails with internal wool lipids

(76) Inventors: Robert James Kelly, Christchurch (NZ); Alisa Dawn Roddick-Lanzilotta, Canterbury (NZ); Sigrid Edith Vorwerk, Christchurch (NZ); Luisa Coderch, Barcelona (ES)

Correspondence Address: BACON & THOMAS, PLLC, 625 SLATERS LANE, FOURTH FLOOR, ALEXANDRIA, VA, 22314, US

(21) Appl. No.: 11/707,044

(22) Filed: Feb. 16, 2007

Related U.S. Application Data
(60) Provisional application No. 60/774,606, filed on Feb. 21, 2006.

(51) Int. Cl. A61K 8/37

(52) U.S. Cl. 424/61; 424/70.7; 424/70.31

(57) ABSTRACT

Animal or human hair or nails, eyelashes or eyebrows are treated with internal wool lipid extract.

### TECHNICAL FIELD

This invention is directed at treatment of hair or nails.

### BACKGROUND OF THE INVENTION

Cosmetic composition containing ceramide is known for application to hair and nails.

For example, Cauwet-Martin et al. U.S. Pat. Nos. 5,830,481 and 6,039,962 are directed to compositions comprising a ceramide and protein or peptide with at least one fatty chain for shampoos, rinses, styling, hair setting, blow drying, fixing, hair dyeing, bleaching, permanent waving, straightening, or to be applied before or after shampooing, dyeing, bleaching, permanent-waving or straightening.

Moreover, Gaetani et al. U.S. Pat. No. 6,846,940 teaches use of novel ceramide compounds for use for hair care, nail care, in a nail varnish, and in a mascara.

Heretofore, it has not been suggested that internal wool lipids are useful as a source of ceramides and other lipids for use on hair or nails or eyebrows or eyelashes despite the fact the internal wool lipids besides having multiple functionality because of including multiple lipid compounds, would be better accepted than lipid ingredients of synthetic origins.

### SUMMARY OF THE INVENTION

One embodiment herein, denoted the first embodiment, is directed to a method of treating animal or human hair comprising the step of applying to said hair, a hair treating composition comprising from 0.05 to 0.30 wt percent internal wool lipid extract (neat basis) or from 0.5 to 2% by weight internal wool lipid extract liposome suspension (10 mg lipid/ml basis).

Another embodiment herein, denoted the second embodiment, is directed to treating human or animal nails, comprising the step of applying to said nails, a nail treating composition comprising from 0.05 to 0.30 wt percent internal wool lipid extract (neat basis) or from 0.5 to 2% by weight internal wool lipid extract liposome suspension (10 mg lipid/ml basis).

Still another embodiment herein, denoted the third embodiment, is directed to a method for shaping and filling in an eyebrow, comprising the step of applying to said eyebrow an eyebrow pencil composition comprising from 0.05 to 0.30 wt percent internal wool lipid extract (neat basis) or from 0.5 to 2% by weight internal wool lipid extract liposome suspension (10 mg lipid/mg basis).

Yet another embodiment herein denoted the fourth embodiment, is directed to a method of thickening and lengthening eyelashes comprising the step of applying to said eyelashes a mascara composition comprising from 0.05 to 0.30 wt percent internal wool lipid extract (neat basis) or from 0.05 to 2% by weight internal wool lipid extract liposome suspension (10 mg lipid/ml basis).

As used herein the term “internal wool lipid extract” without reference to a liposome, means lipids internal to wool (associated with the cell membrane complex, which occurs between corticle cells of keratin fibers) obtained from the wool by extraction after removal of surface lipids and thus excludes lipids found on the surface of wool fibers (referred to as lanolin). The internal wool lipid extracts herein contain from 13 to 35% by weight free fatty acid, 5 to 30% by weight cholesterol, at least 15% by weight ceramides, e.g., 15 to 50% by weight ceramides, e.g., at least 25% by weight ceramides, and 20 to 40% by weight other lipids (cholesterol esters, mono-, di- and triglycerides, derivatives of oxidated cholesterol, cerebrosides, cholesterol sulfate, etc). The ceramides are fatty amides of sphingosine or phytosphingosine and are usually grouped into six types as described in Dowling, D. T., The Journal of Investigative Dermatology 84, 410-412 (1983), the whole of which is incorporated herein by reference. The lipid contents are determined by means of thin layer chromatography (TLC) coupled to a flame ionization detector (FID) system.

The term “internal wool lipid extract liposome suspension” is used herein to mean a microscopic vesicle made by a method comprising forming a suspension of internal wool lipid extract in a salt solution and sonicating at elevated temperature and then letting stand at room temperature.

The term “treating” as used herein means application to cause chemical and/or physical changes to the recipient of the application.

### DETAILED DESCRIPTION

The internal wool lipid extracts for use herein are obtained as described in Coderch, L., et al., J. Am. Oil Chem. Soc. 72, 715-720 (1995), the whole of which is incorporated herein by reference, or as described hereinafter.

Said extracts are obtained from wool fibers of sheep of different breeds and varieties, such as Spanish Merino, South African Merino, New Zealand Merino, Australian Merino and Russian Merino, other wool breeds and varieties including the U.S. wool breeds Bluefaced Leicester, Cotswold, Delaine Merino, Icelandic, Karakul, Leicester Longwool, Rambouillet, Shetland and Targhee, by a process comprising steps of removing surface lipids and subjecting the surface lipid free fibers to extraction.

The step of surface lipid removal is readily carried out, for example, by washing with aqueous detergent containing solution, or by treatment with dry cleaning agents as described in Saville, U.S. Pat. No. 3,619,116. A working example of surface lipid removal is provided in Working Example I hereinafter.

The fibers substantially free of surface lipids including lanolin are subjected to extraction to separate the internal lipids from the fibers. This extraction can be carried out by Soxhlet extraction with an azeotropic mixture of chloroform and methanol as described in Coderch, L., et al., J. Am. Oil Chem. Soc. 72, 715-720 (1995), also cited above. A working example of Soxhlet extraction is provided in Working Example II hereinafter. Said extraction can also be carried out with CO₂ under supercritical conditions for the CO₂. A working example of supercritical extraction is set forth in Working Example III hereinafter. In general, the technique of supercritical extraction with CO₂ provides greater ceramide content than the Soxhlet extraction technique, but the yield is smaller.

Either of the aforementioned methods for extraction has the advantage that the physical structure of the wool fibers is not substantially affected, maintaining its characteristics of resistance and elasticity. The extracted wool therefore retains its usefulness for subsequent industrial processing in, for example, the manufacture of textiles.

The internal wool lipid extract product can be formulated neat, i.e., after removal of solvent, or in the form of an oily solution in physiologically acceptable lipophilic solvent, e.g., vegetable oil or acetylated monoglyceride.

The internal wool lipid extracts can be readily and advantageously formed into liposome form, for formulation. Liposome formation can be carried out as described in Coderch, L., et al., J. Am. Oil Chem. Soc. 73, 1713-1718 (1996), the whole of which is incorporated herein by reference. Liposome formation is readily carried out, for example, by dissolving internal wool lipid extract in solvent, evaporating the solvent to form a film of the extract on a receiver, suspending the extract in a salt (e.g., NaCl) solution, e.g., to obtain a lipid concentration of 10 mg lipid/ml, sonicating at a temperature greater than the phase transition temperature of the lipids of the extract, then obtaining liposomes of selected size by extruding through filters. To associate active agent with the liposome, said agent is dissolved/dispersed in the aqueous phase (salt solution), to which the internal wool lipid extract is added, e.g., in the case of protein active agent in a concentration of 1 μg/μg of lipid prior to the sonication. Active agent that is advantageous for the compositions herein is, e.g., S-sulfonated protein (e.g., S-sulfonated keratin intermediate filament protein(s) (SIFP) and/or S-sulfonated keratin high sulfur protein(s) (SHSP) made, for example, as described in Example 3 of WO 03/011894 A1) or keratin peptides made, for example, as described in Example 4 of WO 03/011894 A1. The liposome form is advantageous compared to neat internal wool lipid extract or solution of internal wool lipid extract from the standpoints of stability, ease of handling and ease of formulation (no association with emulsifier or other component is required as is desirable for neat or solution of internal wool lipid extract, for compatibility with other ingredients of a formulation) and having the characteristic of more effective delivery so that even though the liposome provides less internal wool extract in a formulation than neat internal wool extract it is equally or more efficacious. When associated with another active ingredient, the liposomes facilitate the delivery of said other ingredient, e.g., onto the hair. In a preferred method, the liposomes are formulated present in aqueous suspension, e.g., suspension in an aqueous solution of sodium chloride, and the suspension is added into the formulation gently at the end of the formulation process so the spheres constituting the liposomes remain intact.

For formulation, typically an admixture of aqueous phase ingredients and an admixture of lipophilic (hydrophobic) phase ingredients are made up, and the phases are admixed sometimes with first admixing of surfactant ingredients with the aqueous phase ingredients, with any pH and viscosity adjustment at the end of formulation. When liposomes are included, they are preferably added toward the end of formulating with gentle mixing.

We turn now to the first embodiment herein directed to method of treating human or animal hair.

The internal wool lipid extract ingredient (neat, solution, liposomal form) provides improved physical strength, appearance, feel, body, response to moisture, moisture retention, responses to chemical and environmental insult, and improved response to aqueous and lipophilic treatments including dyes, reductive treatments and other agents commonly applied to the hair. When applied to the hair, the internal wool lipid extract (neat, solution, liposomal form) repair to some extent damage that has been caused by chemical or environmental insult, in particular, damage that has occurred to the cell membrane complex of the hair fiber. The internal wool lipid extract (neat, solution, liposomal form) provides a barrier on hair surface improving moisture retention and regulation properties of the hair.

Tests show that the internal wool lipid extracts (neat, solution, liposomal form) when formulated, on application to hair, improve the mechanical properties of both undamaged and damaged hair. Compositions for use herein on hair show excellent capacity for reinforcing the protecting lipid barrier of human hair and measurably improve the degree of hydration.

The compositions herein used for treatment of hair of humans include hair conditioning compositions, shampoo compositions, leave on conditioner compositions, styling gel compositions, hair straightening (relaxing) compositions, and post chemical treatment (perming, relaxing, bleaching, coloring) conditioning compositions, hair masks (nourishing and moisturizing treatment for dry and damaged hair that untangles hair and gives it extra shine) and texturizing and styling products with internal wool lipid extract supplements.

The compositions herein for use on non-human animals (e.g., dogs, cats, horses) include, for example, shampoos, and styling/grooming products.

The compositions used herein for hair care can also be in the form of those used for topical application to the hair, such as pomades, creams, emulsions, solutions, etc., which may be prepared by association of internal wool lipid extract (neat, solution, liposomal form) with physiologically acceptable excipients and other components of suitable finish, by formulation methods known to those skilled in the art such as mixing and dissolving to form the types of compositions including additives and auxiliary components as described in Cauwet-Martin et al., U.S. Pat. No. 5,830,481.

The proportions of internal wool lipid extract in hair treating compositions herein are illustrated in working examples hereinafter.

The compositions of the first embodiment provide more protection and/or repair on exposure to harmful agents compared to the same composition without internal wool lipid extract ingredient.

We turn now to the second embodiment herein which is directed to treating human or animal nails. The internal wool lipid extract (neat, solution, liposome form) component is advantageously formulated into a nail varnish or nail treatment composition to furnish nail strengthening and other advantages including protection, flexibility, shine and repair.

We turn now to the third embodiment herein directed to a method for shaping or filling in an eyebrow. The internal wool lipid extract (neat, solution, liposome form) component in an eyebrow pencil composition provides the benefits of protection and repair.

We now turn to the fourth embodiment herein directed to a method for thickening or lengthening eyelashes, the internal wool lipid extract (neat, solution, liposome form) component in a mascara composition provides the benefits of protection and repair.

The invention herein is illustrated by the following working examples.

### EXAMPLE I

### Removal of Surface Lipids from Wool

Wool is obtained from living sheep by physical shearing and then the surface fats, also called lanolin, are removed by means of an industrial washing, generally in washing sets consisting of 6 troughs, using a mixture of sodium carbonate and non-ionic surfactant oxyethylenate with 8 to 9 moles of ethylene oxide as detergent. Once the wool has been delanolinized, it is submitted to a treatment of combing the fibers and elimination of mechanical impurities and, then, to soaking in distilled water for a short period of time, in order to eliminate part of the dust and other fine particles contained therein. Finally, wool fibers are dried at room temperature and humidity, finishing the conditioning thereof with a period of 24 hours at 20° C. and a RH of 60%.

### EXAMPLE II

### Extraction of Internal Wool Lipids From Wool Fibers Free of Surface Lipids By Soxhlet Extraction

12g of conditioned wool obtained from the method of Example I are weighed and introduced into a Soxhlet fitted with a cellulous cartridge to prevent the passage of any foreign particles still remaining in the wool. The extractor solvent is a mixture of chloroform/methanol (79:21) with a bath ratio of 1/30, which implies a solvent volume of 360 mL. The extraction is performed at 72° C. for 5 hours, a time period equivalent to six cycles of the Soxhlet. The distillate with the lipid extract is evaporated using a rotavapor apparatus, made up to 10 mL in a solution of chloroform/methanol (2:1) and stored at refrigeration temperature (4° C. ). Then, 1 mL of solution is evaporated to determine, using a gravimetric method, the amount extracted.

By means of the procedure presented above, five varieties of merino wool are extracted which provide the following extraction yields, in which the percentage is expressed by weight of internal wool lipids (IWL) obtained with respect to the initial weight of the wool fiber extracted:

TABLE 1

| | |
|---|---|
| Spanish | 0.947% |
| South African | 0.916% |
| New Zealand | 1.037% |
| Australian | 1.012% |
| Russian | 1.415% |

For a quantitative analysis of the IWLs obtained, a thin layer chromatography (TLC) technique is used coupled to a flame ionization detector (FID) in the automatic apparatus latroscan™ (Labtron Lab. Inc., Tokyo, Japan). The lipid extracts are deposited directly (1.6 mu L) with an automatic depositor Sample spotter SES 3202/IS-01, Germany) on silica columns 10 cm long (Silica /gel SIII Chromarods) and are eluted vertically with three mixtures of solvents in order of decreasing polarity: 1) twice with chloroform/methanol/water (57:12:0.6) up to 2.5 cm; 2) hexane/diethyl ether/formic acetate (50:20:0.3) up to 8 cm; and 3) hexane/benzene (35:35) up to 10 cm. After each elution, the solvent is removed by heating at 60° C. followed by cooling in a chamber at room temperature and drying. The same procedure is applied to the standards: palmitic acetate, cholesterol and ceramide III to thus determine their calibration curves and enabling subsequent quantification of each component. The standards are acquired from Sigma Co. (St. Louis, Mo.) in analytic grade and stored in chloroform/methanol.

The results obtained with the five samples of wool extracted are presented in Table 2 in which said results are expressed as percentages by weight with respect to total weight of IWL extract.

TABLE 2
Chemical composition of the IWLs obtained by means of
extraction procedure with Soxhlet.

| Variety of merino | Free fatty acids | Cholesterol | Ceramides | Rest (*) |
|---|---|---|---|---|
| wool | (%) | (%) | (%) | (%) |
| Spanish | 20.09 | 21.25 | 31.88 | 26.79 |
| South African | 22.44 | 24.74 | 29.30 | 23.52 |
| New Zealand | 32.57 | 17.70 | 27.65 | 22.08 |
| Australian | 23.39 | 25.28 | 25.98 | 25.35 |
| Russian | 13.30 | 12.71 | 34.89 | 39.10 |

(*) Remaining lipid components not individually quantified, which are found in lower proportions, such as cholesterol esters, mono-, di- and triglycerides, derivatives of oxidated cholesterol, cerebrosides, cholesterol sulphate, etc.

### EXAMPLE III

### Extraction of Wool Lipid Extracts From Wool Fibers Free of Surface Lipids by Supercritical Extraction with CO₂

The apparatus used to perform the supercritical extraction has a double syringe pump (SFC-3000; Fisons, Milan, Italy) that distributes the CO₂ (SFE grade 99.998%, Praxir Espa a, Barcelona, Spain) and the modifier of polarity (methanol in analytical grade, Merck, Darmstad, Germany), in the desired proportions. The function of the modifier is to increase the capacity of extraction and dissolving of CO₂. The flow of liquid is adjusted by means of a regulating valve (Hoke Inc., Creskill, N.J., USA) to 1.5-2.0 ml.min, measured from the LCD of the pump, keeping the temperature at 100° C.

4.8 g of wool already processed in accordance with the procedure of Example I are introduced into the cylinder where the extraction takes place in supercritical conditions for CO₂, these being 340 atmospheres, 20 min, at a temperature of 100° C. and 10% methanol. The total volume of liquid used for the extraction is 4-5 times the volume of the cell. The extract is collected in a 15-mL vial sealed beforehand with septum (Supelco, Bellefonte Pa., USA) which has an outlet to eliminate decompressed CO₂. The extract is concentrated until dry under nitrogen flow, or by heating to 60° C. , in order to eliminate excess solvent. The IWL extracted is quantified using a gravimetric method and resuspended in chloroform/methanol 2:1 (1 mL), and kept under refrigeration.

Following the extraction procedure described above, two varieties of merino wool are extracted which provide the following extraction yields, in which the percentage is expressed by weight of IWL extract with respect to the initial weight of the wool fiber extracted.

TABLE 3

| | |
|---|---|
| Spanish | 0.290% |
| Zealand | 0.355% |

For quantitative analysis of the IWL obtained, the chromatographic technique already described in Example II is used, and the results obtained with the two samples of wool extracted are presented in Table 4, in which said results are expressed as percentages by weight with respect to total weight of IWL extract.

TABLE 4
Chemical composition of the IWLs obtained by means of
supercritical extraction with CO₂ (SFE)

| Variety of merino | Free fatty acids | | Ceramides | Rest (*) |
|---|---|---|---|---|
| wool | (%) | Cholesterol (%) | (%) | (%) |
| Spanish | 17.75 | 7.41 | 45.29 | 29.55 |
| New Zealand | 26.02 | 7.75 | 29.73 | 36.50 |

(*) Remaining lipid components not individually quantified, which are found in lower proportions, such as cholesterol esters, mono-, di- and triglycerides, derivatives of oxidated cholesterol, cerebrosides, cholesterol sulphate, etc.

As can be seen from comparison of the data obtained in Examples II and III, the method of extraction using the Soxhlet provides much better extraction yields, but the SFE method has a smaller affinity for the extraction of cholesterol and a somewhat greater yield with respect to the extraction of the ceramides.

### EXAMPLE IV

### Obtaining Liposomes

An IWL extract dissolved in chloroform/methanol 2:1 (v/v) from either Example II or III is evaporated to dryness under a nitrogen atmosphere, and the film formed on the walls of the recipient is resuspended with a suitable volume of NaCl solution at 0.9% by weight to obtain a suspension of lipid concentration of 10 mg/ml. The formation of multi-lamina liposomes is carried out by sonicating the sample at 65° C. , which is a temperature greater than that of phase transition of the implicated lipids, for 15 minutes in a Labsonic™ 1510 sonicator (B. Braum, Germany).

After leaving to stand for 24 hours at room temperature, uni-lamina liposomes of a defined size are obtained by extruding the multi-lamina liposome suspension three times, at 65° C., through filters with a pore size of 800×400×200 nm (Millipore™, Ireland), providing a suspension of liposomes whose size can be checked to be approximately 200 nm by means of the technique of light scattering, using a Autosizer™ photocorrelation Ile spectrometer (Malvern, UK).

In other cases SIFP or SHSP as made in Example 3a of WO 03/011894 A1 is included in the NaCl solution at a level of 1 μg/l of lipid. The product is IWL liposome associated with SIFP in one case and SHSP in the other case.

In another case, keratin peptides as made in Examples 4A, 4b or 4c of WO 011894 A1 are included in the NaCl solution at a level of 1 μg/μl of lipid. The product is IWL liposome associated with keratin peptides.

In the following Examples V-IX, internal wool lipid extract is added in neat form and is referred to as IWL.

### EXAMPLE V

Hair Conditioner (Humans)

| | |
|---|---|
| INCI name | wt % |

| | | |
|---|---|---|
| A | Aqua | to 100% |
| | EDTA | 0.10 |
| | Lactamide MES(and)Acetamide MEA | 2.00 |
| B | Cetyl Alcohol(and)Behentrimonium | 2.00 |
| | Methosulfate(and)Quarternium-33 | |
| | Behentrimonium | 1.50 |
| | Methosulfate(and)Cetyl | |
| | Alcohol(and)Butylene Glycol | |
| | Polawax GP200 | 2.50 |
| | Synthetic Spermacetic Fatty Acid mixed | 1.00 |
| | Esters | |
| | PPG-3 Benzyl Ether Myristate | 2.00 |
| | Avocado Oil | 1.00 |
| | Astrocaryum Murumuru | 0.50 |
| | IWL | 0.25 |
| C | Tocopherol Acetate | 0.50 |
| | Preservative | Qs |
| | Fragrance | Qs |
| | Citric acid | pH 3.5-4.5 |

Preparation Procedure
Combine Part A and mix until dissolved, heat to 70° C.
Combine Part B and heat to 70° C.
Add Part A into B with constant mixing (Ultra Turrax), mix for 10 minutes.
Cool to 40-45° C. and add Part C.
Mix for 10 minutes until uniform.
Adjust pH with citric acid to 3.5-4.5.
   Application
Wash and rinse hair thoroughly. Dispense conditioner onto the palm of the hand and massage into the hair from root to tip. Rinse off with warm water.

### EXAMPLE VI

Shampoo (Humans)

| A | Aqua | qs to 100 |
|---|---|---|
| | Guar Hydroxypropyltrimonium | 0.20 |
| | Chloride | |
| | Disodium EDTA | 0.10 |
| B | Sodium Laureth Sulfate | 17.00 |
| | Disodium Laureth | 14.00 |
| | Sulfosuccinate(30%) | |
| | Cocamide DEA(70%) | 1.50 |
| | Cocoamidopropyl Betaine(30%) | 3.00 |
| C | IWL | 0.25 |
| | Tocopherol Acetate | 0.10 |
| | Preservative | Qs |
| | Glycol Distearate(and)Laureth- | 3.00 |
| | 4(and)Cocamidopropyl Betaine | |
| | PEG-7 Glyceryl Cocoate | 0.50 |
| | Perfume | Qs |
| | Sodium chloride | 1.40 |

Preparation Procedure
Combine Part A with good mixing. Heat Part A to 45° C. Add sodium laureth sulphate and stir until dissolved. Once dissolved, add rest of part B in order with good stirring.
Ensure all ingredients are dissolved. Add part C. Mix perfume with PEG-7 Glyceryl cocoate separately before adding. Adjust viscosity (2000 cps) with salt and pH to 6.5 if necessary.
   Application
Dispense shampoo onto the palm of the hand and massage into the hair until it is fully covered with lather. Rinse off with warm water.

### EXAMPLE VII

Leave On Conditioner (Humans)

| | | |
|---|---|---|
| A | Aqua | to 100% |
| | Hydroxyethylcellulose | 0.10 |
| B | Polyvinyl Pyrrolidone | 0.10 |
| | Vitamine E | 0.20 |
| | Polyquarternium-7 | 0.50 |
| | IWL | 0.10 |
| | Glycerine | 3.00 |
| | EDTA tetrasodium salt | 0.10 |
| | Arnica Extract | 0.10 |
| | D-Panthenol | 1.00 |
| | Propylenglycol(and)diazolidinyl urea | 0.50 |
| | (and) methylparaben (and) | |
| | propylparaben | |
| C | Citric acid | pH 5.5 to 6.5 |

Preparation Procedure
Combine Part A with good mixing ca. 20 minutes, heat to 40° C. till everything is dissolved.
Add Part B in order with constant stirring.
Adjust pH to 5.5-6.0 with citric acid.
   Application
Apply to damp or dry hair from a pump bottle. Style as usual.

### EXAMPLE VIII

Styling Gel (Humans)

| | | |
|---|---|---|
| A | Aqua | to 100% |
| | Carbomer | 0.20 |
| B | Polyacrylate-14 | 2.70 |
| | Sorbitol | 0.40 |
| | Panthenol | 0.50 |
| | Propylene Glycol | 0.10 |
| | Disodium EDTA | 0.10 |
| | PEG-33(and)PEG-8 | 0.20 |
| | Dimethicone(and)PEG-14 | |
| | Aminomethyl Propanol(95%) | pH 6.8-7.2 |
| C | IWL | 0.20 |
| | Fragrance | qs |
| | Preservative | qs |

Preparation Procedure
Sift the amount of carbopol into the vortex of water. Mix for 20-30 minutes or until completely hydrated.
Add the ingredients of Part B one at a time, mixing until uniform between each 20 addition.
Neutralize to pH 6.8-7.2 using aminomethyl propanol.
Add Part C and stir carefully until homogeneous.
   Application
Dispense required quantity onto hands, work through dry or damp hair with the fingers, style as usual.

### EXAMPLE IX

Hair Relaxer (Straightener) Cream (Humans)

| | | |
|---|---|---|
| A | Petrolatum | 40.00 |
| | Cetearyl Alcohol | 8.00 |
| | Lanolin | 4.00 |
| | PEG-75 Lanolin | 3.00 |
| | Sodium Stearate | 1.20 |
| B | Aqua | to 100% |
| | Sodium Hydroxide | 0.30 |
| C | IWL | 0.20 |
| | Fragrance | Qs |

Preparation Procedure
Mix Part A together in a stainless steel container and heat to 70° C.
Mix Part B together in a stainless steel container and heat to 70° C.
Add Part B into A, mixing for 10 minutes. Cool to 50° C. and add Part C.
   Application
Apply to dry hair, carefully covering all regions. Leave for 10-20 minutes and carefully comb with a wide toothed comb to straighten. Rinse thoroughly and condition. Avoid overexposure of the skin and avoid eye contact.

In the following examples X-XIV, internal wool lipid extract is added in the form of liposomes made as described in Example IV and is referred to as IWL liposome. The formulation and application is the same as for the formulation and application for the corresponding IWL extract (neat) compositions above except that the IWL liposome is added near the end of the preparation with only gentle mixing.

### EXAMPLE X

Hair Conditioner (Humans)

| | | |
|---|---|---|
| A | Aqua | to 100% |
| | EDTA | 0.10 |
| | Lactamide MES(and)Acetamide | 2.00 |
| | MEA | |
| B | Cetyl | 2.00 |
| | Alcohol(and)Behentrimonium | |
| | Methosulfate(and)Quarternium-33 | |
| | Behentrimonium | 1.50 |
| | Methosulfate(and)Cetyl | |
| | Alcohol(and)Butylene Glycol | |
| | Proprietary Blend | 2.50 |
| | Synthetic Spermacetic Fatty Acid | 1.00 |
| | mixed Esters | |
| | PPG-3 Benzyl Ether Myristate | 2.00 |
| | Avocado Oil | 1.00 |
| | Astrocaryum Murumuru | 0.50 |
| C | Tocopherol Acetate | 0.50 |
| | IWL liposome* | 2.0 |
| | Preservative | Qs |
| | Fragrance | Qs |
| | Citric acid | pH 3.5-4.5 |

*including IWL liposome including SIFP or keratin peptides.

Preparation Procedure
Combine Part A and mix until dissolved, heat to 70° C.
Combine Part B and heat to 70° C.
Add Part A into B with constant mixing (Ultra Turrax), mix for 10 minutes.
Cool to 40-45° C. and add Part C.
Mix for 10 minutes until uniform.
Adjust pH with citric acid to 3.5-4.5.

### EXAMPLE XI

Shampoo (Humans)

| A | Aqua | qs to 100 |
|---|---|---|
| | Guar Hydroxypropyltrimonium | 0.20 |
| | Chloride | |
| | Disodium EDTA | 0.10 |
| B | Sodium Laureth Sulfate | 17.00 |
| | Disodium Laureth | 14.00 |
| | Sulfosuccinate(30%) | |
| | Cocamide DEA(70%) | 1.50 |
| | Cocoamidopropyl Betaine(30%) | 3.00 |
| C | IWL liposome* | 2.00 |
| | Tocopherol Acetate | 0.10 |
| | Preservative | Qs |
| | Glycol Distearate(and)Laureth- | 3.00 |
| | 4(and)Cocamidopropyl Betaine | |
| | PEG-7 Glyceryl Cocoate | 0.50 |
| | Perfume | Qs |
| | Sodium chloride | 1.40 |

*including IWL liposome including SIFP or keratin peptides.

Preparation Procedure
Combine Part A with good mixing. Heat Part A to 45° C. Add sodium laureth sulphate and stir until dissolved. Once dissolved, add rest of part B in order with good stirring.
Ensure all ingredients are dissolved. Add part C. Mix perfume with PEG-7 Glyceryl cocoate separately before adding. Adjust viscosity (2000 cps) with salt and pH to 6.5 if necessary.

### EXAMPLE XII

Leave On Conditioner (Humans)

| | | |
|---|---|---|
| A | Aqua | to 100% |
| | Hydroxyethylcellulose | 0.10 |
| B | Polyvinyl Pyrrolidone | 0.10 |
| | Vitamine E | 0.20 |
| | Polyquarternium-7 | 0.50 |
| | IWL liposome | 2.00 |
| | Glycerine | 3.00 |
| | EDTA tetrasodium salt | 0.10 |
| | Arnica Extract | 0.10 |
| | D-Panthenol | 1.00 |
| | Propylenglycol(and)diazolidinyl urea | 0.50 |
| | (and) methylparaben (and) | |
| | propylparaben | |
| C | Citric acid | pH 5.5 |
| | | to 6.5 |

Preparation Procedure
Combine Part A with good mixing ca. 20 minutes, heat to 40° C. till everything is dissolved.
Add Part B in order with constant stirring.
Adjust pH to 5.5-6.0 with citric acid.

### EXAMPLE XIII

Styling Gel (Humans)

| | | |
|---|---|---|
| A | Aqua | to 100% |
| | Carbomer | 0.20 |
| B | Polyacrylate-14 | 2.70 |
| | Sorbitol | 0.40 |
| | Panthenol | 0.50 |
| | Propylene Glycol | 0.10 |
| | Disodium EDTA | 0.10 |
| | PEG-33(and)PEG-8 | 0.20 |
| | Dimethicone(and)PEG-14 | |
| | Aminomethyl Propanol(95%) | pH 6.8-7.2 |
| C | IWL liposome | 2.00 |
| | Fragrance | Qs |
| | Preservative | Qs |

Preparation Procedure
Sift the amount of carbopol into the vortex of water. Mix for 20-30 minutes or until completely hydrated.
Add the ingredients of Part B one at a time, mixing until uniform between each addition.
Neutralize to pH 6.8-7.2 using aminomethyl propanol.
Add Part C and stir carefully until homogeneous.

### EXAMPLE XIV

Hair Relaxer (Straightener) Cream (Humans)

| | | |
|---|---|---|
| A | Petrolatum | 40.00 |
| | Cetearyl Alcohol | 8.00 |
| | Lanolin | 4.00 |
| | PEG-75 Lanolin | 3.00 |
| | Sodium Stearate | 1.20 |
| B | Aqua | to 100% |
| | Sodium Hydroxide | 0.30 |
| C | IWL liposome | 2.00 |
| | Fragrance | qs |

Preparation Procedure
Mix Part A together in a stainless steel container and heat to 70° C.
Mix Part B together in a stainless steel container and heat to 70° C.
Add Part B into A, mixing for 10 minutes. Cool to 50° C. and add Part C.

### EXAMPLE XV

Dog Shampoo

| | | |
|---|---|---|
| A | Aqua | qs |
| | Sodium Laureth Sulfate | 10.00 |
| | Sodium Methyl Cocoyl Taurate | 8.00 |
| B | Cocamidopropyl Betaine | 12.00 |
| | Glycol distearate,Cocamide | 4.00 |
| | DEA,Sodium Lauryl ethoxysulfate | |
| | Polyquarternium-7 | 0.60 |
| | IWL liposome | 2.00 |
| C | Citric acid | pH 4.5 |
| D | Sodium Chloride | 1.80 |
| E | Preservative | qs |

Procedure
Combine Part A ingredient with good mixing, ensure all ingredients are dissolved.
Add Part B in order.
Adjust the pH with Part C.
Adjust the viscosity with Part D.
Add Part E.

### EXAMPLE XVI

Dog Styling Gel

| | | |
|---|---|---|
| A | PPG-3 benzyl Ether Myristate | 10.00 |
| | Squalene | 10.00 |
| | Oleth-3 | 8.00 |
| | Oleth-3 Phophate | 7.00 |
| | Oleth-5 | 5.00 |
| B | | qs |
| | Propylene Glycol | 10.00 |
| | PPG-5 Ceteth-20 | 2.00 |
| | Glycerin | 1.00 |
| C | IWL liposome | 2.00 |
| | Preservative | qs |

Procedure
Combine Part A and B separately and heat to 65° C.
Add Part B to A while mixing (Ultra Turax).
Cool to 45° C. add Part C in order. Stir gently.
Pour into containers before the gel reaches its set point.

### EXAMPLE XVII

Horse Shampoo

| | | |
|---|---|---|
| A | Aqua | qs |
| | Disodium EDTA | 0.10 |
| B | Sodium Laureth Sulfate | 15.00 |
| | Aqua/Amine Oxide | 4.00 |
| | Cocamidopropyl Betaine | 8.00 |
| | Glyco Distearate/Laureth- | 4.00 |
| | 4/Cocamidopropyl Betaine | |
| C | Di-PPG-2 Myreth-10 Adipate | 2.00 |
| | IWL liposome | 2.00 |
| D | Arnica Montana Flower Extract | 1.00 |
| | Tocopherol Acetate | 1.00 |
| | Preservative | qs |
| E | Sodium Chloride 20% sol. | 0.5 |
| F | Lactic Acid | pH 6.5-6.8 |

Procedure
Combine and dissolve Part A.
Add Part B in order and stir until dissolved.
Mix Part C separately and add to AB.
Add Part D while stirring.
Adjust viscosity with Part E.
Adjust pH with Part F.

### EXAMPLE XVIII

Nail Cream (strengthening)

| A | Aqua | Qs |
|---|---|---|
| | Glycerine | 3.00 |
| B | Polawax GP200 | 10.00 |
| | Caprylic/Capric Triglyceride | 5.00 |
| | Behentrimonium Methosulfate(and)Cetearyl | 3.00 |
| | Alcohol | |
| | Di-PPG-3 Myristyl Ether Adipate | 1.00 |
| C | IWL liposome | 2.00 |
| | Phenoxyethanol(and)Methylparaben | 1.00 |
| | (and)Ethylparaben(and)Butylparaben | |
| | (and)Propylparaben(and)Isopropyl- | |
| | Paraben | |
| | Chamomilla Recutita | 0.50 |
| | Hamamelis Virginiana | 0.50 |

Procedure
Mix Part A and heat to 65° C.
Mix Part B and heat to 65° C.
Add Part A into B and mix well (Ultra Turrax) for 10 minutes.
Cool to 45° C. and add Part C.
Mix gently for another 10 minutes.

### EXAMPLE XIX

Eyebrow Pencil

| | | |
|---|---|---|
| A | Stearic Acid | 40.00 |
| | Cetyl Alcohol | 10.00 |
| | Hydrogenated vegetable oil | 20.00 |
| B | Black iron oxides | Qs |
| C | IWL liposome | 2.00 |

Procedure
Melt ingredients of Part A and add Part B while stirring.
Cool to 45° C. and add Part C.

### EXAMPLE XX

Mascara

| | | |
|---|---|---|
| A | Sucrose stearate | 4.00 |
| | Polyglyceryl-3 methylglycose | 2.00 |
| | distearate | |
| | Stearyl Alcohol | 1.00 |
| | Candelilla Wax | 5.00 |
| | Carnauba Wax | 1.80 |
| | Cera Alba | 4.30 |
| | Hydrogenated rice bran wax | 5.00 |
| | Adipic acid/diethylene glycol/glycerin | 5.00 |
| | crosspolymer | |
| B | IWL liposome | 2.00 |
| C | Iron oxide | 10.00 |
| D | Butandiol-1,3 | 3.00 |
| | Triethanolamine | 1.50 |
| | Acrylates/octylacrylamide copolymer | 5.00 |
| E | Aqua | qs |
| F | Preservatives | qs |

Procedure
Combine Part A and heat to 70° C. Add and dissolve B in A.
Add and disperse C in AB. Stirring until completely dissolved.
Add DE to ABC. Cool to 30° C. Add Part F.
Adjust viscosity with stearyl alcohol.
   Variations

The foregoing description of the invention has been presented describing certain operable and preferred embodiments. It is not intended that the invention should be so limited since variations and modifications thereof will be obvious to those skilled in the art, all of which are within the spirit and scope of the invention.

What is claimed is:

1. A method of treating human or animal hair comprising the step of applying to said hair, a hair treating composition comprising from 0.05 to 0.30 wt percent internal wool lipid extract (neat basis) or from 0.5 to 2% by weight internal wool lipid extract liposome suspension (10 mg lipid/ml basis).

2. The method of claim 1 where the application is to human hair.

3. The method of claim 1 where the application is to animal hair.

4. A method of treating human or animal nails, comprising the step of applying to said nails, a nail treating composition comprising from 0.05 to 0.30 wt percent internal wool lipid extract (neat basis) or from 0.5 to 2% by weight internal wool lipid extract liposome suspension (10 mg lipid/ml basis).

5. A method for shaping and filling in an eyebrow, comprising the step of applying to said eyebrow, an eyebrow pencil composition comprising from 0.05 to 0.3 wt percent internal wool lipid extract (neat basis) or from 0.5 to 2% by weight internal wool lipid extract liposome suspension (10 mg lipid/ml basis).

6. A method for thickening and lengthening eyelashes comprising the step of applying to said eyelashes from 0.05 to 0.30 wt percent internal wool lipid extract (neat basis) or from 0.5 to 2% by weight internal wool lipid extract liposome suspension (10 mg lipid/ml basis).
